(19) United States
(12) Patent Application Publication
(10) Pub. No.: US 2025/0049448 A1
(43) Pub. Date: Feb. 13, 2025

(54) TUNNEL DRILLING AIMER AND ISO-ANGLE USER INTERFACE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian William QUIST, Salem, NH (US); Geoffrey Ian KARASIC, Raynham, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/761,873

(22) Filed: Jul. 2, 2024

Related U.S. Application Data
(60) Provisional application No. 63/518,627, filed on Aug. 10, 2023.

(51) Int. Cl. A61B 17/17

(52) U.S. Cl. CPC A61B 17/1764

(57) ABSTRACT

In one embodiment of the present disclosure a method may include presenting, on a user interface, a single aimer indicator to identify when a first parameter of a tool is satisfied, wherein the first parameter pertains to a first axis associated with the tool. The method may include modifying a first aspect of the single aimer indicator to identify when a second parameter of the tool changes, wherein the second parameter pertains to a second axis associated with the tool. The method may include determining when a threshold associated with the second parameter is satisfied. Responsive to determining the threshold associated with the second parameter is satisfied, the method may include modifying a second aspect of the single aimer indicator to represent the tool is in coaxial alignment.

### CROSS-REFERENCE TO RELATED APPLICATIONS

This application claims the benefit of U.S. Provisional Application No. 63/518,627 filed Aug. 10, 2023 and titled “Tunnel Drilling Aimer and Iso-Angle User Interface.” The provisional application is incorporated by reference herein as if reproduced in full below.

### BACKGROUND

The anterior cruciate ligament (ACL) serves as the primary mechanical restraint in the knee to resist anterior translation of the tibia relative to the femur. Similarly the posterior cruciate ligament (PCL) serves as a mechanical restraint to resist posterior translation of the tibia relative to the femur. These cruciate ligaments contribute significantly to knee stability, and ACL injury is quite common. Most ACL injuries are complete tears of the ligament.

As ACL injuries occur often in patients that are young and active, reconstruction of the ACL is performed to enable return to activity. The goal is to restore stability of the knee and reduce the chances of further damage to the meniscus and articular cartilage that may lead to degenerative osteoarthritis. Reconstruction may consist of placement of a substitute graft (e.g., autograft from either the central third of the patellar tendon or the hamstring tendons). The ends of the graft are placed into respective tunnels prepared through the femur and the tibia. The ends of the graft may be attached using interference screws or a suspensory fixation device like the ENDOBUTTON™ brand fixation devices manufactured by Smith & Nephew of Andover, Massachusetts, USA.

One step in performing reconstruction surgery of the ACL includes drilling a femoral socket with a reamer. Other types of reconstruction surgery also include drilling a tunnel to enable a surgeon to tie down tissue or pass through a graft to restore an anatomical function. Orientation of a tunnel is inherently a three-dimensional problem, where users align the tip of an object to an intended trajectory as well as the orientation of a shaft to a trajectory. Further, other challenges for drilling a tunnel may include accurately approximating a knee angle that corresponds to an exit-point associated with placing the tunnel.

### SUMMARY

In some examples, a method may include presenting, on a user interface, a single aimer indicator to identify when a first parameter of a tool is satisfied, wherein the first parameter pertains to a first axis associated with the tool. The method may include modifying a first aspect of the single aimer indicator to identify when a second parameter of the tool changes, wherein the second parameter pertains to a second axis associated with the tool. The method may include determining when a threshold associated with the second parameter is satisfied. Responsive to determining the threshold associated with the second parameter is satisfied, the method may include modifying a second aspect of the single aimer indicator to represent the tool is in coaxial alignment.

In some examples, a surgical controller includes a processor configured to couple to a display device and a memory coupled to the processor, the memory storing instructions that, when executed by the processor, cause the processor to present, on a user interface on the display device, a single aimer indicator to identify when a first parameter of a tool is satisfied, wherein the first parameter pertains to a first axis associated with the tool. The instructions further cause the processor to modify a first aspect of the single aimer indicator to identify when a second parameter of the tool changes, wherein the second parameter pertains to a second axis associated with the tool. The instructions further cause the processor to determine when a threshold associated with the second parameter is satisfied, and responsive to determining the threshold associated with the second parameter is satisfied, modify a second aspect of the single aimer indicator to represent the tool is in coaxial alignment.

In some examples, a tangible, non-transitory computer-readable storage medium stores instructions that, when executed, cause a processing device to present, on a user interface on the display device, a single aimer indicator to identify when a first parameter of a tool is satisfied, wherein the first parameter pertains to a first axis associated with the tool. The instructions further cause the processor to modify a first aspect of the single aimer indicator to identify when a second parameter of the tool changes, wherein the second parameter pertains to a second axis associated with the tool. The instructions further cause the processor to determine when a threshold associated with the second parameter is satisfied, and responsive to determining the threshold associated with the second parameter is satisfied, modify a second aspect of the single aimer indicator to represent the tool is in coaxial alignment.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows a conceptual drawing of a surgical site with various objects within the surgical site tracked, in accordance with at least some embodiments;

FIG. 6 is an example video display showing portions of a femur and having visible therein a bone fiducial, in accordance with at least some embodiments;

FIG. 7 is an example video display showing intraoperative changes to a tunnel path, in accordance with at least some embodiments;

FIG. 8 is an example user interface including an aimer indicator representing axial alignment via fill of a circle, in accordance with at least some embodiments;

FIG. 9 is an example graphical representation of an aimer indicator that changes based on alignment, in accordance with at least some embodiments;

FIG. 10 is another example graphical representation of an aimer indicator that changes based on alignment, in accordance with at least some embodiments;

FIG. 11 is an example user interface including an aimer indicator representing axial alignment via bars, in accordance with at least some embodiments;

FIG. 12 is another example graphical representation of an aimer indicator that changes based on alignment, in accordance with at least some embodiments;

FIG. 13 is another example graphical representation of an aimer indicator that changes based on alignment, in accordance with at least some embodiments;

FIG. 14 is an example user interface including a three-dimensional trajectory alignment of hardware in anatomy, in accordance with at least some embodiments;

FIG. 15 is an example user interface including targets for where a tool should contact a bone, in accordance with at least some embodiments;

FIG. 16 is an example image depicting iso-angle feedback, in accordance with at least some embodiments;

FIG. 17 is another example image depicting iso-angle feedback, in accordance with at least some embodiments;

FIG. 18 is an example flow chart of a method for using a single aimer indicator to represent when a tool is in coaxial alignment, in accordance with at least some embodiments;

FIG. 19 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counter bore may thus define an internal shoulder. However, the term “counterbore” shall not be read to imply any method of creation. A counterbore may be created in any suitable way, such as drilling, boring, laser drilling, or casting.

The terms “input” and “output” when used as nouns refer to connections (e.g., electrical, software), and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections. In systems implemented in software, these “inputs” and “outputs” define parameters read by or written by, respectively, the instructions implementing the function.

“Controller” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), or a field programmable gate array (FPGA), configured to read inputs and drive outputs responsive to the inputs.

“Real-time” may refer to an action, operation, method, function, instruction, etc. being performed within 2 seconds. “Near real-time” may refer to an action, operation, method, function, instruction, etc. being performed between 2 seconds and 1 minute.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems for a tunnel drilling aimer and isometric angle user interface. Sports medicine procedures may involve the use of creating tunnels in bones to enable a surgeon to tie down tissue or pass through a graft to restore an anatomical function. As discussed above, orientation of a tunnel may be a three-dimensional problem, where users align a tip of an object to an intended trajectory as well as the orientation of a shaft to a trajectory. Because users have different capabilities with spatial reasoning, there is a need to provide different visual representations of a targeting system while drilling tunnels. This is because certain visual feedback enhances user interfaces to provide enhanced user experiences using computing devices, thereby providing a technical improvement. Further, certain users prefer certain visual feedback based on user preference.

Further, arthroscopic navigation may be enhanced by the disclosed embodiments that may include placement of a femoral footprint and tunnel trajectory for ACL reconstruction. For example, in some embodiments, a guide-wire aimer may be placed to place a femoral ACL pin that is subsequently over-drilled. In some embodiments, surgeons planning an arthroscopic navigation solution may be provided visual feedback of a region of exit-point placements that may be achievable when placing a tunnel. When planning arthroscopic navigation solutions, surgeons may face challenges related to accurate approximation of knee angles that correspond to exit-point output. In some embodiments, an overlay of determined or calculated knee flexion angles (depicted as isometric-knee (“iso-knee”) flexion angle lines) may be presented on a user interface to enable a surgeon to better approximate patient positioning. Patient position knee flexion angle may be provided in degrees of flexion.

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Thus, the description and developmental context shall not be read as a limitation of the applicability of the teachings. In order to orient the reader, the specification first turns a description of the knee.

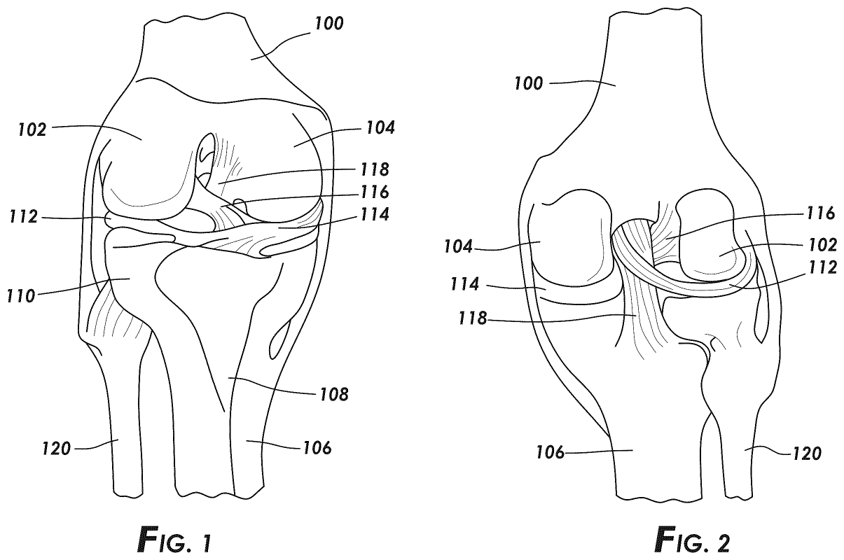

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy's tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

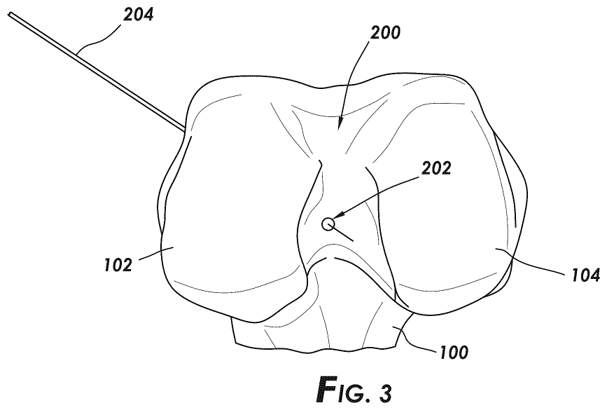

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon's selection for location of the port through the skin, and through which the various instruments are inserted into the knee, it may be difficult to reach the attachment location of the native ACL to the femur 100.

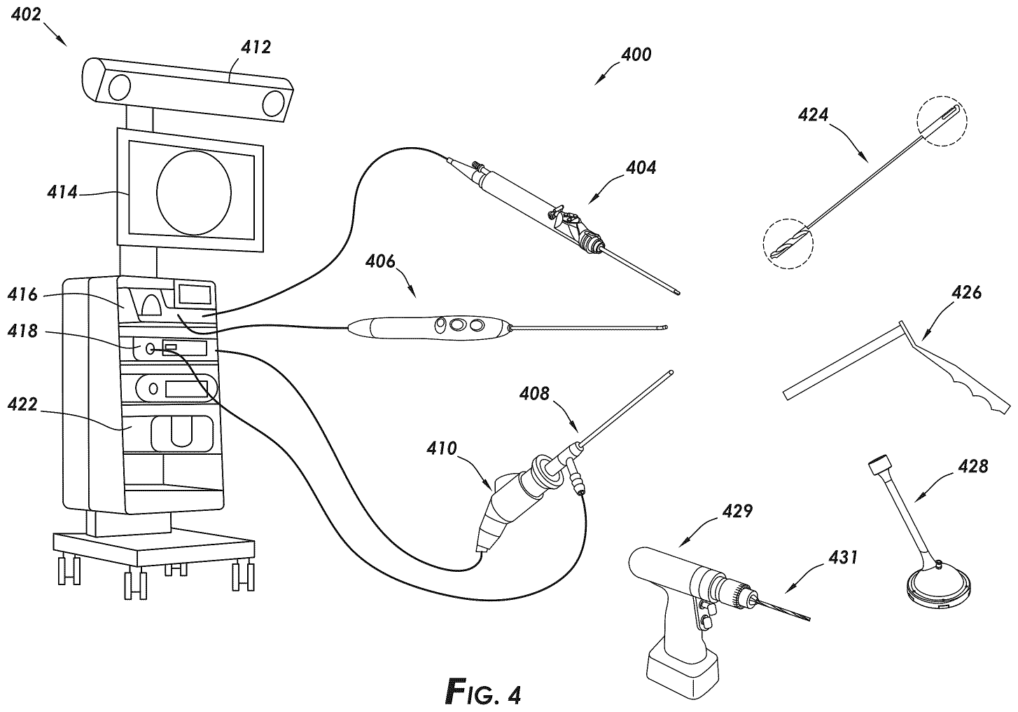

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define inside aperture 202 within the femoral notch 200, the inside aperture 202 closer to the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the drill wire 204 is closely aligned with a planned-tunnel path, the femoral tunnel is created by boring or reaming with another instrument (e.g., a reamer) that may use the drill wire 204 as a guide. In some cases, a socket or counterbore is created on the intercondylar notch side to accommodate the width of the graft that extends into the bone, and that counterbore may also be created using another instrument (e.g., reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The further examples discussed below are thus based on an inside-out procedure, but such should not be read as a limitation. The specification now turns to an example surgical system.

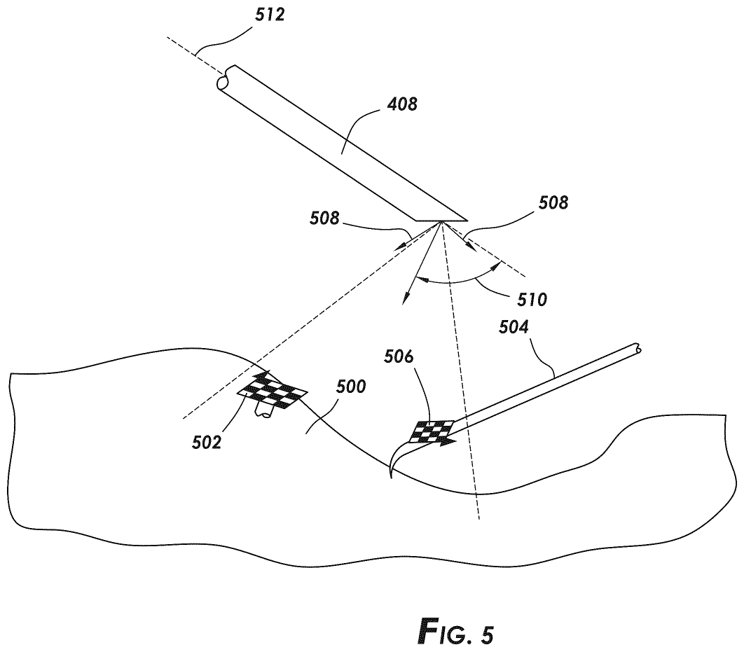

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 not only provides light to the arthroscope 408 and displays images received from the camera head 410, but also implements various additional aspects, such as displaying a single aimer indicator that changes aspects depending on when a tool is in coaxial alignment, displaying an overlay of iso-angles representing knee angles to achieve a target exit angle, and so forth. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 may then assess whether the pilot tunnel matches or closes matches the planned-tunnel path. If the pilot tunnel is deemed sufficient, then the drill wire 424 may be used as a guide for creating the full-diameter throughbore for the tunnel, or alternatively, for creating a counterbore stemming from the intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As will be discussed in greater detail below, the calibration assembly 428 may be used to detect optical distortion in images received by the surgical controller 418 through the arthroscope 408 and attached camera head 410.

Additional tools and instruments will be present, such as a drill 429 for drilling with the drill wire 424, various reamers 431 for creating the throughbore and counterbore aspects of the tunnel, and various tools for suturing and anchoring the graft in place. Some of these additional tools and instruments are not shown so as not to further complicate the figure.

The specification now turns to a workflow for an example ACL repair. The workflow may be conceptually divided into a preoperative planning and intraoperative repair. The intraoperative repair workflow may be further conceptually divided into optical system calibration, model registration, intraoperative tunnel-path planning, intraoperative and tunnel creation. Each will be addressed in turn.

### Preoperative Planning

In accordance with various examples, an ACL repair starts with preoperative imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The discussion that follows assumes MRI imaging, but again many different types of preoperative imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of ACL repair and specifically selecting a tunnel path through the femur, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native ACL to the femur within the femoral notch. In some cases, selecting the entry location within the femoral notch may involve use of a Bernard & Hertel Quadrant or grid placed on a fluoroscopic image, or placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the preoperative planning may comprise: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia.

In some embodiments, using the three-dimensional bone model, the surgeon may select a planned value for a socket or counterbore depth of a reamer into a bone. The surgeon may select the socket or counterbore depth such that a socket is not created that is too long, thereby resulting in a thin bone wall that may be too close to the cortex. A bone wall that is thin and too close to the cortex may result in a failure of the ACL reconstruction. In addition, using the three-dimensional bone model, the surgeon may select a tunnel throughbore diameter, a counterbore diameter, and the like. One or more reamers may be selected based on the diameters of the tunnel throughbore diameter and/or the counterbore diameter.

In some embodiments, the three-dimensional bone model may include a virtual representation of the imaged bones of the patient mapped into a three-dimensional coordinate space (e.g., virtual space). The dimensions, size, density, length, width, depth, height, shape, position, configuration, orientation, and the like of the bones may be accurately represented in the three-dimensional coordinate space.

Other surgical parameters may also be selected during the preoperative planning, such as desired post-repair flexion, and the like, but those additional surgical parameters are omitted so as not to unduly complicate the specification.

### Intraoperative Repair

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient's skin, and in fact before the patient is wheeled into the surgical room. Nevertheless, such steps and procedures are considered intraoperative as they take place in the surgical setting and with the surgical equipment and instruments used to perform the actual repair.

The example ACL repair is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the bone within the three-dimensional coordinate space of the view of the arthroscope, the location of the reamer within the three-dimensional coordinate space of the view of the arthroscope, and location of the various instruments (e.g., the drill wire 424, the aimer 426) within the three-dimensional coordinate space of the view of the arthroscope. The specification turns to brief description of such tracking techniques.

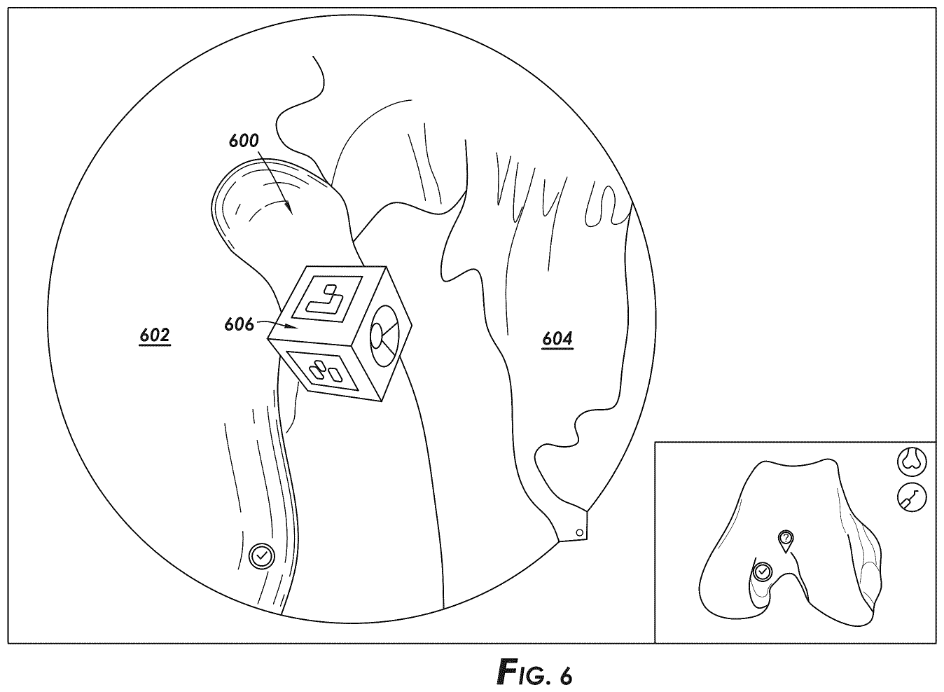

FIG. 5 shows a conceptual drawing of a surgical site with various objects within the surgical site. In particular, visible in FIG. 5 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light. In the example of FIG. 5, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410 (FIG. 4). The images detected by the capture array within the camera head 410 are sent electronically to the surgical controller 418 (FIG. 4) and displayed on the display device 414 (FIG. 4). In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location the port created through the skin of the patient. In the example of FIG. 5, the view angle 510 of the arthroscope 408 forms a non-zero angle to the longitudinal central axis 512 of the arthroscope 408.

Still referring to FIG. 5, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410 (FIG. 4).

The probe fiducial 506 is shown as a planar element attached to the touch probe 504. The touch probe 504 may be used, as discussed more below, to “paint” the surface of the bone 500 as part of the registration of the bone 500 to the three-dimensional bone model, and the touch probe 504 may also be used to indicate revised-tunnel entry locations in the case of intraoperative changes to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the point of the touch probe 504, may be determined from images captured by the arthroscope 408 and attached camera head 410 (FIG. 4).

Other instruments within the view of the arthroscope 408 may also have fiducials, such as the drill wire 424 (FIG. 4) and aimer 426 (FIG. 4), but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426 (FIG. 4)), the location may be tracked by an optical array coupled to the aimer and viewed through the camera 412 (FIG. 4), such as a stereoscopic camera. The location within the three-dimensional coordinate space of the camera 412 is then transformed into the three-dimensional coordinate space of the view of the example arthroscope to determine location of the distal end within the surgical site.

The images captures by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process.

### -Optical System Calibration

In example systems, prior to use within each surgical procedure, the endoscopic optical system is calibrated to account for the various optical distortions. In particular, various embodiments comprise a system for calibrating the endoscopic optical system. Referring again to FIG. 4, the example system comprises the surgical controller 418, the arthroscope 408, the camera head 410, and the calibration assembly 428. In particular, the calibration may comprise placing the arthroscope 408 into the calibration assembly 428. The calibration assembly 428 holds the arthroscope 408 in a fixed relationship to a calibration target on an inside surface of the calibration assembly 428. Once the distal end of the arthroscope 408 is within the calibration assembly 428, the example method comprises capturing a plurality of images of the calibration target, with each image captured at a unique rotational relationship between the camera head 410 and the arthroscope 408, with the unique rotational relationships relative to a longitudinal central axis of the arthroscope 408. Using the plurality of images, the example surgical controller 418 creates a characterization function that characterizes optical distortion between the calibration target and the capture array within the camera head 410. The characterization function may include a calibration for determining orientation of fiducial markers visible within the surgical site (e.g., bone fiducial 502, probe fiducial 506) by way of the arthroscope 408 and attached camera head 410.

### -Model Registration and Human-In-the-Loop Registration Verification

The next example step in the intraoperative procedure is the registration of the bone model(s) created during the preoperative planning. That is, during the preoperative planning stage, preoperative imaging (e.g., MRI) of the knee takes place, including the relevant anatomy like the lower portion of the femur, the upper portion of the tibia, and the articular cartilage. The preoperative imaging can be segmented such that a volumetric model or three-dimensional model of the anatomy is created. More specifically to the example of ACL repair, and specifically selecting a tunnel path through the femur, a three-dimensional bone model of the lower portion of the femur is created during the preoperative planning. Further, a depth of the tunnel path may be selected and a diameter of the tunnel may be selected. The depth and diameter may be represented in the volumetric or three-dimensional model (e.g., a virtual space including axes and coordinates (e.g., X, Y, Z)).

During the intraoperative repair, the three-dimensional bone models are provided to the surgical controller 418. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient's skin, the surgical controller 418 also receives video images of the femur. In order to relate the three-dimensional bone model to the images received by way of the arthroscope 408 and camera head 410, the surgical controller 418 registers the three-dimensional bone model to the images of the femur received by way of the arthroscope 408 and camera head 410.

In accordance with example methods, a fiducial marker or bone fiducial (e.g., bone fiducial 502 of FIG. 5) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial is placed within the intercondylar notch superior to or above the expected location of the tunnel through lateral condyle.

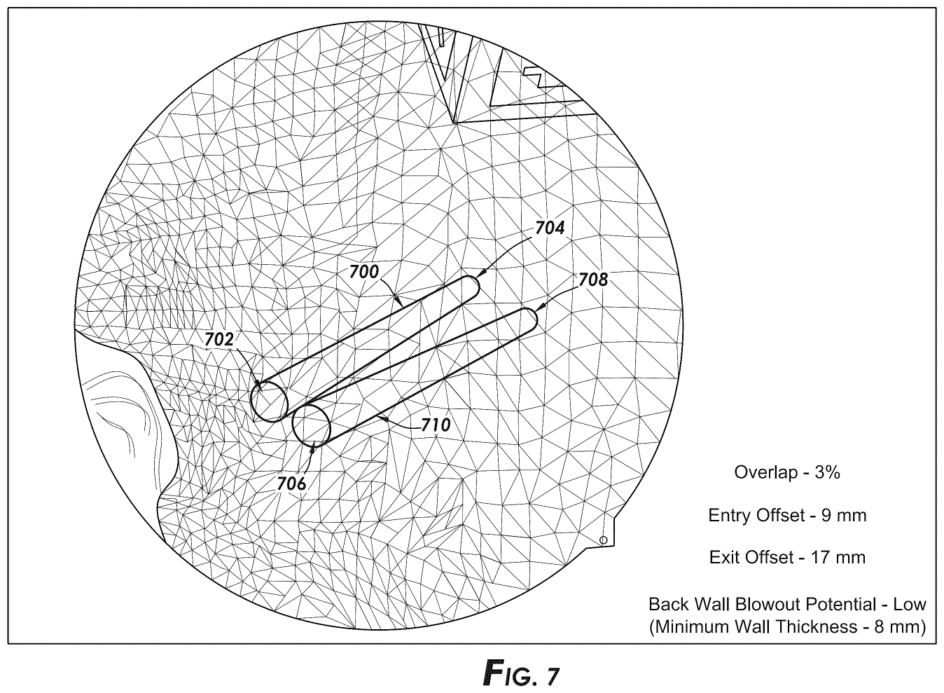

FIG. 6 is an example video display showing portions of a femur and a bone fiducial. The display may be shown, for example, on the display device 414 (FIG. 4) associated with the device cart 402 (FIG. 4), or any other suitable location. In particular, visible in FIG. 6 is a femoral notch or intercondylar notch 600, a portion of the lateral condyle 602, a portion of the medial condyle 604, and an example bone fiducial 606. The bone fiducial 606 is a fiducial comprising a cube member. Of the six outer faces of the cube member, the bottom face is associated with an attachment feature (e.g., a screw). The bottom face will be close to or abut the bone when the bone fiducial 606 is secured in place, and thus will not be visible in the view of the arthroscope 408 (FIG. 4). The outer face opposite the bottom face includes a placement feature used to hold the bone fiducial 606 prior to placement, and to attach the bone fiducial 606 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 606 represents a fixed location on the outer surface of the bone in the view of the arthroscope 408, even as the position of the arthroscope 408 is moved and changed relative to the bone fiducial 606. Initially, the location of the bone fiducial 606 with respect to the three-dimensional bone model is not known to the surgical controller 418, hence the need for the registration of the three-dimensional bone model.

In or order to relate or register the bone visible in the video images to the three-dimensional bone model, the surgical controller 418 (FIG. 4) is provided and thus receives a plurality of locations of an outer surface of the bone. For example, the surgeon may touch a plurality of locations using the touch probe 504 (FIG. 5). As previously discussed, the touch probe 504 comprises a probe fiducial 506 (FIG. 5) visible in the video images captured by the arthroscope 408 (FIG. 4) and camera head 410 (FIG. 4). The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” locations of the outer surface of the bone relative to the bone fiducial 606. Given that the touch probe 504 is a relatively inflexible instrument, in other examples the tracking of the touch probe 504 may be by optical tracking of an optically-reflective array outside the surgical site (e.g., tracking by the camera 412 (FIG. 4)) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone. In some embodiments, other modes of capturing the shape of the bone relative to the bone fiducial 606 may include using computed tomography (CT) and/or fluoroscopy with or without contrast (e.g., a substance that is injected into an intravenous line or taken by mouth to enable the body part (e.g., bone tissue and structure) being examined to be seen more clearly). CT scans may use an X-ray beam that traverses a body part externally in a circular motion to obtain many different views of the same body part and those images may be used to generate a two-dimensional form of the body part on a video display. Fluoroscopy may refer to imaging technique that uses a fluoroscope to obtain real-time moving images of a body part of the patient. CT and/or fluoroscopy may enable obtaining updated images of the outer surface of the bone relative to the bone fiducial 606. In some embodiments, another mode of capturing the shape of the bone relative to the bone fiducial 606 may include using an externally tracked ultrasound probe. An external tracker may be used to identify a position and orientation of images obtained by an ultrasound probe that may be merged to identify the outer surface of the bone relative to the bone fiducial 606.

A video display may be displayed during a registration procedure showing portions of a femur, a bone fiducial, and an overlaid representation of the three-dimensional bone model. The display may be shown, for example, on the display device 414 (FIG. 4) associated with the device cart 402 (FIG. 4), or any other suitable location. In order to address potential invalid registrations between the three-dimensional bone model and the bone visible in the images, in example methods and systems an initial registration between the bone visible in the video images and the three-dimensional bone model is verified using a human-in-the-loop process. More particularly, in example cases, after receiving the plurality of locations of an outer surface of the bone visible in the images and performing an initial registration to the three-dimensional bone model, the surgical controller 418 displays a representation of the three-dimensional bone model overlaid on portions of the bone visible in the video images, the displaying and overlaying on the display device 414, or other suitable location. That is, the example surgical controller 418 overlays a representation of the three-dimensional bone model (e.g., by way of a polygon mesh, or mesh model) on the display of the images captured by the arthroscope 408 and camera head 410, wherein the rotational and translational alignment of the three-dimensional bone model is correlated to the bone visible in the video images as anchored by the bone fiducial 606.

The surgeon, in turn, visually studies the overlaid representation of the three-dimensional bone model relative to the underling bone visible in the video images to determine whether the registration process was correct. More particularly, the surgeon visually compares the overlaid representation of the three-dimensional bone model to the portion of the bone visible in the video images to determine whether the three-dimensional bone model sufficiently matches the bone visible in the video images. Much like the registration process itself, the human-in-the-loop verification of registration is a non-deterministic exercise. Slight variances between the three-dimensional bone model and the bone visible in the video images may be tolerated, yet nevertheless the registration process may be considered correct in the sense that the three-dimensional bone model may be reliably used to help guide placement of the tunnel path (e.g., here the femoral tunnel path), or assist the surgeon in intraoperative changes to the planned-tunnel path. In such cases, the surgeon may provide the surgical controller 418, and the surgical controller 418 may thus receive, an indication that the three-dimensional bone model is correctly registered to the bone visible in the video images.

On the other hand, if the overlay of the three-dimensional bone model shows misalignment with the bone visible in the video images, the surgeon may elect to restart the registration process, such as by providing to the surgical controller 418, and the surgical controller 418 receiving again, a plurality of locations of the outer surface of the bone. The surgical controller 418 may then perform anew the registration procedure. In other cases, the surgeon may elect to provide additional locations on the outer surface of the bone, and the surgical controller 418 may then perform the registration procedure with the both original locations received and the additional locations received after the overlay process. The process repeats until the surgeon approves the registration. The specification now turns to intraoperative tunnel path planning.

### -Tunnel Path Planning

Using the three-dimensional bone model an operative plan is preoperatively created that comprises a planned-tunnel path through the bone, including locations of the apertures into the bone that define the ends of the tunnel. Further, a depth of the planned-tunnel path is selected and a diameter of the tunnel path is selected.

In some embodiments, when planning a surgical solution, a surgeon may be presented (e.g., on the display device 414) with visual feedback of a region of exit-point placements that are achievable when placing the tunnel. For example, an overlay may be presented on an image of a bone and the overlay may include determined or calculated knee flexion angle, depicted as iso-knee flexion angle lines, to enable the surgeon to better approximate patient positioning.

In some cases, however, the surgeon may elect not to use planned-tunnel path, and thus elect not to use the preoperatively planned entry location, exit location, or both. Such an election can be based any of a number of reasons. For example, intraoperatively the surgeon may not be able to access the entry location for the planned-tunnel path, and thus may need to move the entry location to ensure sufficient access. As another example, during the intraoperative procedure the surgeon may determine that the planned tunnel entry location is misaligned with the attachment location of the native ACL to the femur. Further still, during the intraoperative procedure the surgeon may determine the tunnel entry location is too close to the posterior wall of the femur, increasing the likelihood of a bone chip sometimes referred to as a “back wall blowout.” Regardless of the reason for the election to change the tunnel path, in example systems the surgical controller 418 enables the surgeon to intraoperatively select a revised-tunnel entry, a revised-tunnel exit (if needed), and thus a revised-tunnel path through the bone.

FIG. 7 is an example video display showing intraoperative changes to the tunnel path, in accordance with at least some embodiments. The display may be shown, for example, on the display device 414 (FIG. 4) associated with the device cart 402 (FIG. 4), or any other suitable location. In particular, FIG. 7 shows a portion of the bone in the video images as captured by the arthroscope 408 (FIG. 4) overlaid with the mesh model of the three-dimensional bone model and a preoperatively selected planned-tunnel path 700 comprising a planned-tunnel entry 702 and a planned-tunnel exit 704. However, for any number of reasons, the surgeon may elect to modify the tunnel entry location and/or the tunnel exit location, and thus modify the planned-tunnel path. Thus, the surgeon may provide to the surgical controller 418 (FIG. 4), and thus the surgical controller 418 may receive, a revised-tunnel entry location or just revised-tunnel entry 706. Providing the revised-tunnel entry 706 may comprise the surgeon touching the proposed location on the bone shown in the video images with a tracked tool, such as the touch probe 504 (FIG. 5) or the aimer 426 (FIG. 4). In some cases, the surgeon may select the revised-tunnel entry 706 based solely on what the surgeon sees of the bone shown in the video images. As a more specific example, the surgeon may select and provide the revised-tunnel entry 706 based on a location that can be reached by the aimer 426. In yet still other cases, the surgical controller 418 may generate a simulated fluoroscopic images from the three-dimensional bone model, and project thereon a Bernard & Hertel Quadrant or grid. The surgeon may then select the revised-tunnel entry 706 with the additional guidance provided by the Bernard & Hertel Quadrant.

### -Tunnel Creation

With the revised-tunnel path 710 selected, the next step in the example method is creation of the actual tunnel. In most cases, creating the tunnel is a multistep process involving drilling an initial or pilot tunnel using a drill wire (e.g., drill wire 424 (FIG. 4)), and then using the drill wire as guide wire for one or more reamers to increase the diameter of the pilot tunnel to form the full-diameter actual tunnel through the bone. In some cases the actual tunnel has a counterbore associated with the intercondylar notch to accommodate the width of the autograft, and in such cases an additional reamer may be used to create the counterbore.

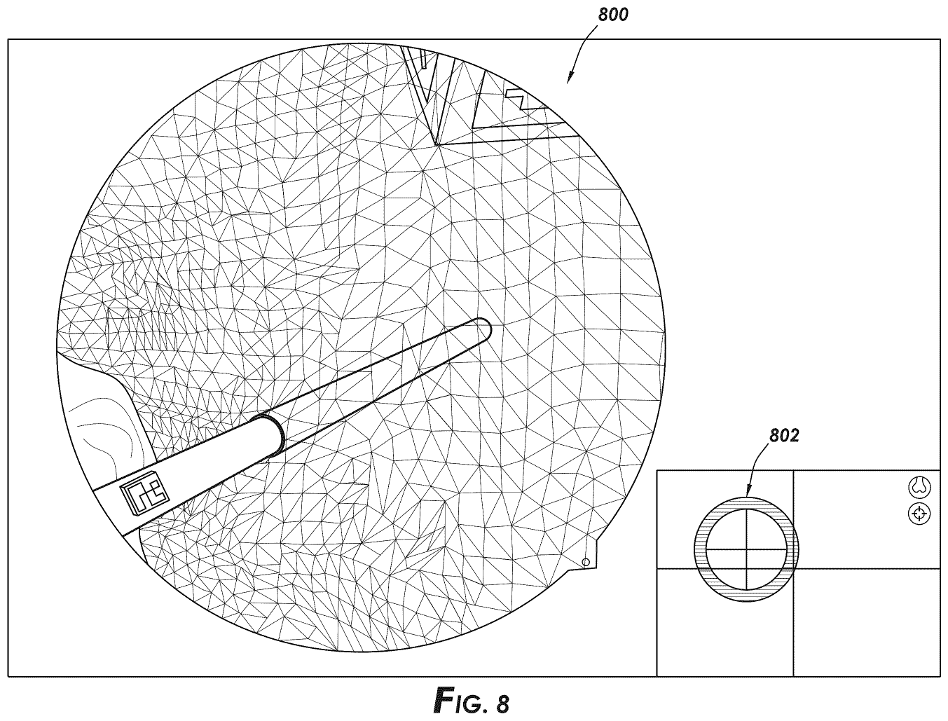

FIG. 8 is an example user interface 800 including a single aimer indicator 802 representing coaxial alignment via fill of a circle, in accordance with at least some embodiments. The user interface 800 may present the single aimer indicator 802 in a lower right corner of a display device 414 (FIG. 4), for example. FIG. 8 shows a portion of the bone in the video images as captured by the arthroscope 408 (FIG. 4) overlaid with the mesh model of the three-dimensional bone model and a preoperatively selected planned-tunnel path. In the depicted embodiment, the single aimer indicator 802 is represented as a cross-hair. The cross-hair represents alignment in a first axis when the tip of a tool is centered over an intended trajectory and alignment in a second axis when the orientation of a shaft of the tool is aligned with the trajectory. An aspect of the single aimer indicator 802 may be modified when alignment in the first axis is achieved. The aspect may include increasing or decreasing a fill of the circle of the cross-hair representing the single aimer indicator 802. For example, as the tip of the tool is aligned with the intended trajectory (which may be referred to as a first parameter), the circle may be increasingly filled or may be decreasingly filled. Additionally, when the orientation of the shaft of the tool is also aligned in a second axis (which may be referred to as a second parameter), a determination may be made whether a threshold associated with the second parameter is satisfied. If the threshold is satisfied, then a second aspect of the single aimer indicator 802 may be modified. For example, the second aspect may include changing a color, flashing a color, or the like of the single aimer indicator 802. Such techniques may enable coaxial aiming of a tool with a target tunnel entry point and trajectory. The second parameter may relate to an angle of the tool's actual trajectory compared to a planned trajectory. As depicted in the embodiment, the single aimer indicator 802 includes a single graphical element that enables depicting coaxially alignment of the tool. Such a technique may enhance the user interface by reducing the graphical elements that are presented, thereby enhancing a user's experience using the computing device and providing a technical improvement.

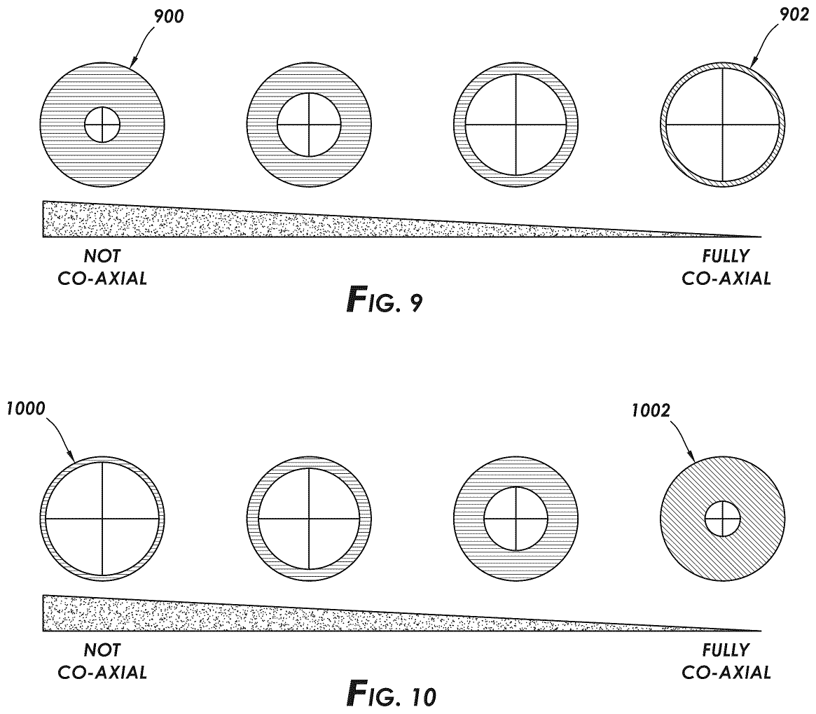

FIG. 9 is an example graphical representation of an aimer indicator that changes based on alignment, in accordance with at least some embodiments. As depicted in the embodiment, the circle of the aimer indicator 900 is filled more completely when one or more axes are misaligned and the thickness of the aimer indicator's 902 circle fill decreases as coaxial alignment improves. When the coaxial alignment satisfies a certain threshold (e.g., greater than a certain percentage of alignment), then another visual cue of the aimer indicator may be performed. For example, the visual cue may include changing a color of the aimer indicator, flashing a color of the aimer indicator, or the like.

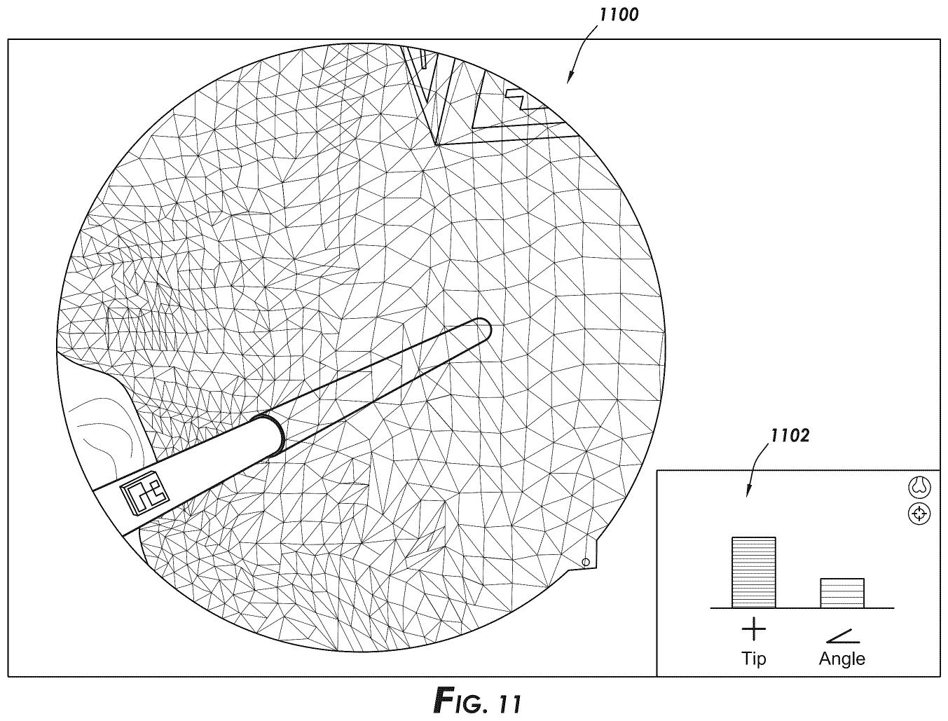

FIG. 10 is another example graphical representation of an aimer indicator that changes based on coaxial alignment, in accordance with at least some embodiments. As depicted in the embodiment, the circle of the aimer indicator 1000 is filled less completely when an axis is misaligned and the thickness of the aimer indicator's 1002 circle fill increases as alignment improves. When the alignment satisfies a certain threshold (e.g., greater than a certain percentage of alignment), then another visual cue of the aimer indicator may be performed. For example, the visual cue may include changing a color of the aimer indicator, flashing a color of the aimer indicator, or the like.

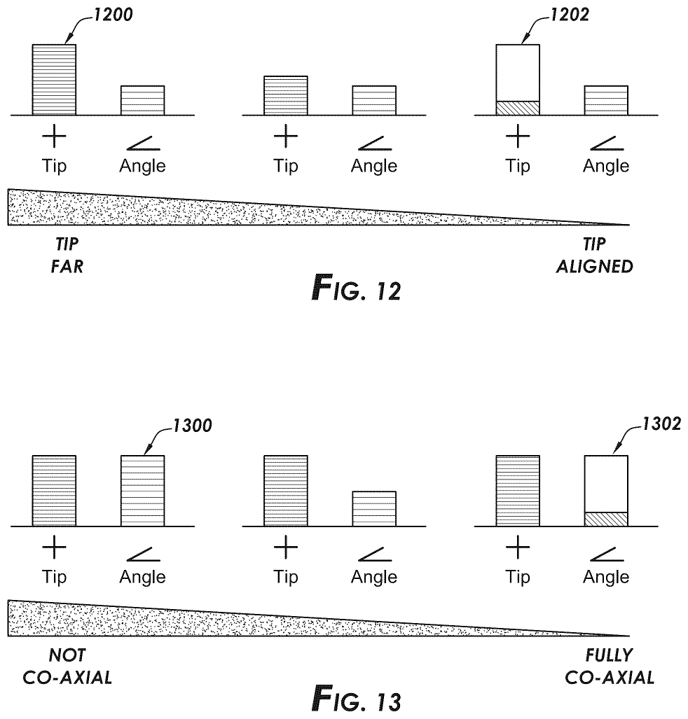

FIG. 11 is an example user interface 1100 including an aimer indicator 1102 representing axial alignment via bars, in accordance with at least some embodiments. FIG. 11 shows a portion of the bone in the video images as captured by the arthroscope 408 (FIG. 4) overlaid with the mesh model of the three-dimensional bone model and a preoperatively selected planned-tunnel path. As depicted, the aimer indicator 1102 is presented in the lower right corner of the user interface 1100. However, it should be understood that the aimer indicator 1102 may be presented in any suitable portion of the user interface 1100. In the depicted embodiment, the tip distance to a target is shown as a scaled bar reflecting the scaled absolute distance in (x, y, z) coordinates. The angular difference between the target trajectory and the actual trajectory is also shown as a scaled bar also reflecting the scaled absolute angular difference between the target vector and the tool vector (theta, phi). The scaled bars' graphical representation may be modified as the tool tip distance from the target increases or decreases and as the angular difference increases or decreases.

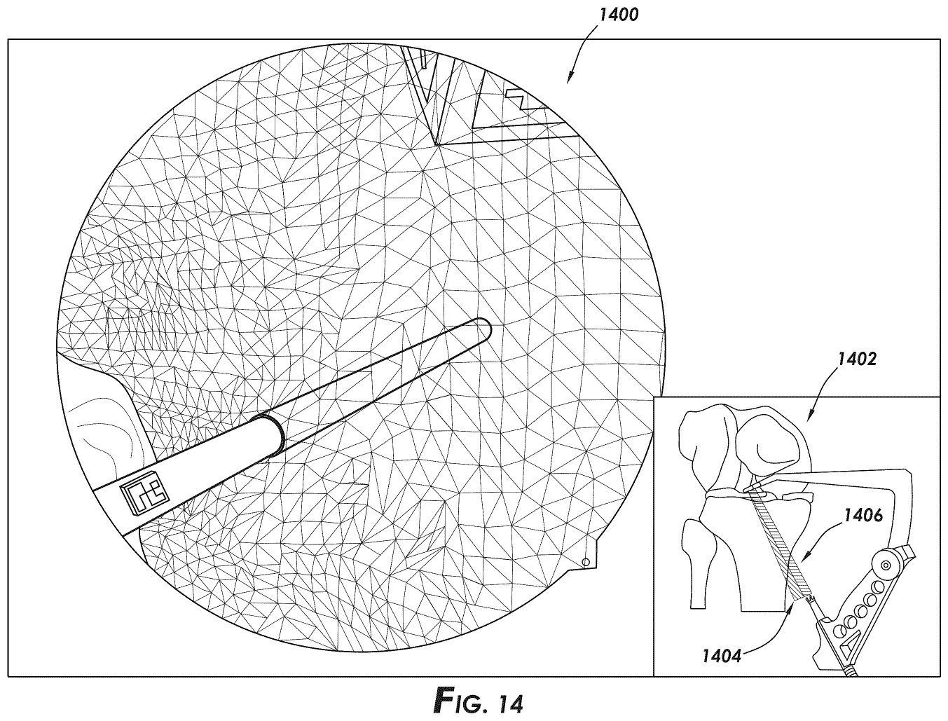

FIG. 12 is another example graphical representation of an aimer indicator that changes based on alignment, in accordance with at least some embodiments. The aimer indicator is represented as scaled bars for both tip axial alignment and angle axial alignment. As depicted, the tip scaled bar 1200 is filled more fully when the tip is misaligned and the fill of the bar decreases as the tip is more axially aligned. When the tip alignment satisfies a threshold amount, another visual cue may be performed, such as by modifying a color of the scaled bar 1202 representing tip axial alignment, flashing a color of the scaled bar representing tip axial alignment, highlighting a border of the scaled bar representing tip axial alignment, or the like.

FIG. 13 is another example graphical representation of an aimer indicator that changes based on alignment, in accordance with at least some embodiments. The aimer indicator is represented as scaled bars for both tip axial alignment and angle axial alignment. As depicted, the angle scaled bar 1300 is filled more fully when the angle axis is misaligned and the fill of the bar decreases as the angle is more axially aligned. When the angle alignment satisfies a threshold amount, another visual cue may be performed, such as by modifying a color of the scaled bar 1202 representing angle axial alignment, flashing a color of the scaled bar representing angle axial alignment, highlighting a border of the scaled bar representing angle axial alignment, or the like.

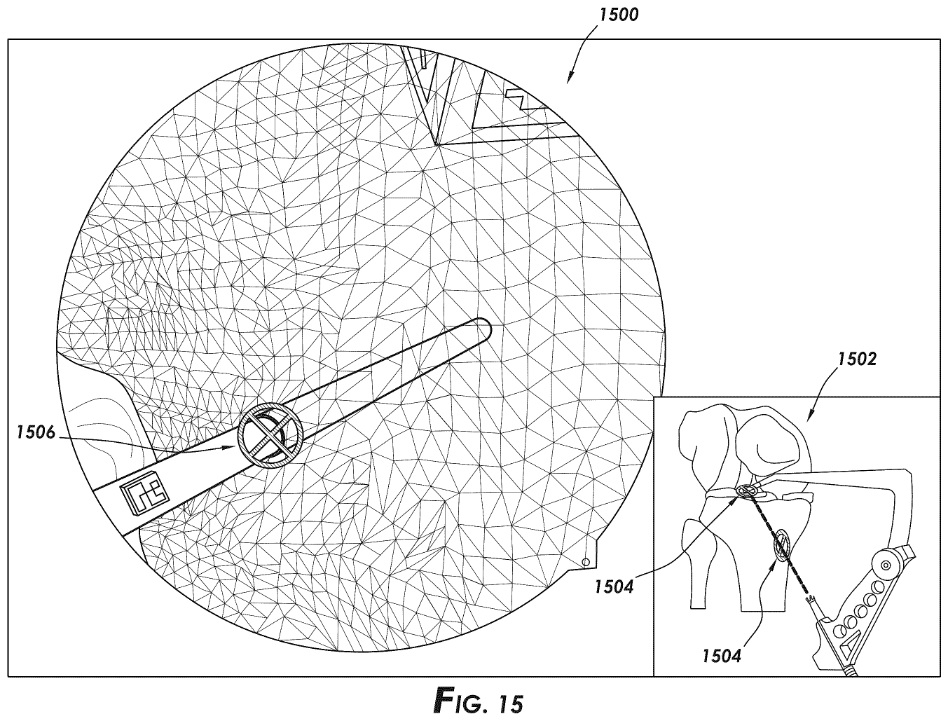

FIG. 14 is an example user interface 1400 including a three-dimensional trajectory alignment of hardware in anatomy, in accordance with at least some embodiments. The user interface 1400 may present the aimer indicator 1402 in a lower right corner of a display device 414 (FIG. 4), for example. FIG. 14 shows a portion of the bone in the video images as captured by the arthroscope 408 (FIG. 4) overlaid with the mesh model of the three-dimensional bone model and a preoperatively selected planned-tunnel path. The aimer indicator 1402 may represent a planned trajectory 1404 in a first color (e.g., green) and a navigated trajectory 1406 in a second color (e.g., blue). As the trajectories become more aligned, an onscreen notification may be presented on a user interface, opacity of the planned tunnel may change, color of the planned tunnel may change, or the like.

In some embodiments, three multiple perspectives may be presented simultaneously. For example, three orthogonal perspectives aligned to the standard planes of anatomy may aid in informing the user which way to manipulate the tool to achieve coaxial alignment.

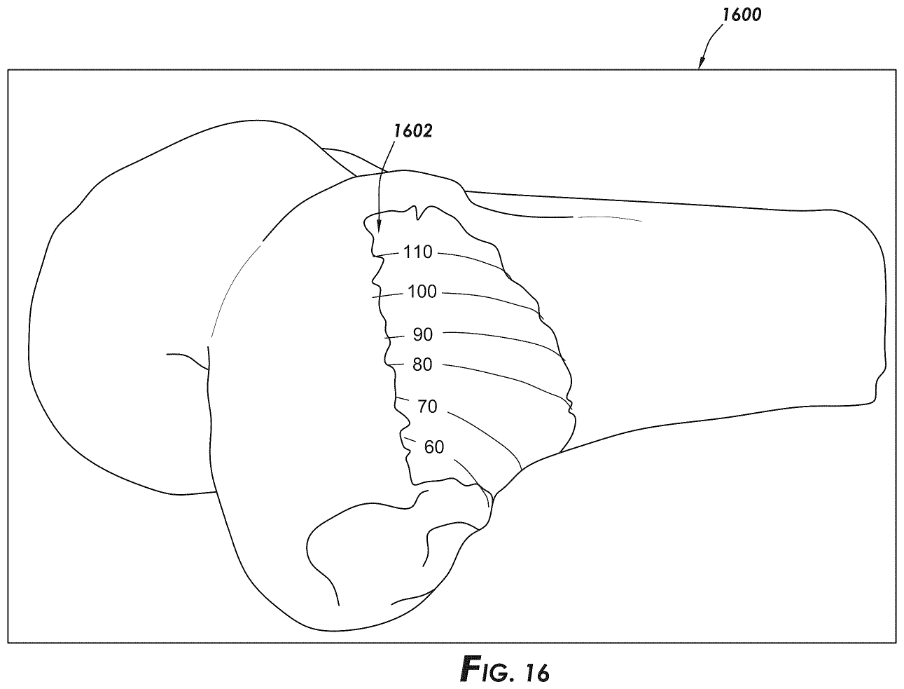

FIG. 15 is an example user interface 1500 including targets 1504 for where a tool (e.g., aimer) should contact a bone, in accordance with at least some embodiments. FIG. 15 shows a portion of the bone in the video images as captured by the arthroscope 408 (FIG. 4) overlaid with the mesh model of the three-dimensional bone model and a preoperatively selected planned-tunnel path. As depicted, a portion 1502 of the user interface 1500 may include an image of a bone and targets 1504 for two bone contacting points. By aligning the two bone contacting points on the aimer to the planned targets on the bone, the navigated trajectory may match the planned trajectory. Further, an intra-articular target 1506 is depicted on the arthroscopic image in the user interface 1500. The intra-articular target 1506 may be placed near an entry point for the tunnel.

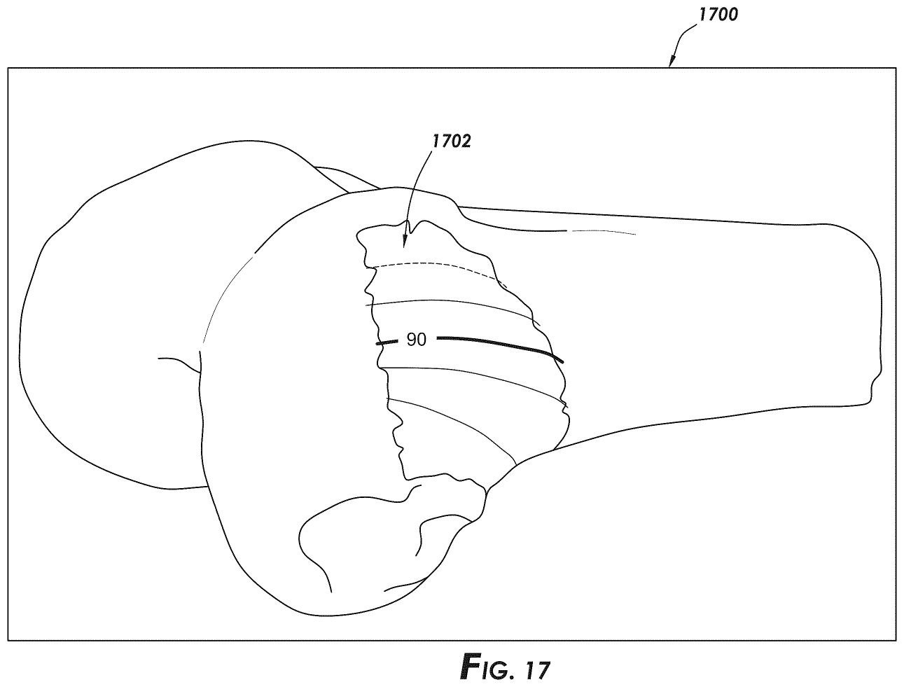

FIG. 16 is an example image 1600 depicting iso-angle feedback 1602, in accordance with at least some embodiments. As depicted, the iso-angle feedback 1602 may be presented to the surgeon (e.g., on display device 414) when planning the potential trajectory of the exit point for drilling a tunnel. Each iso-angle may represent a knee angle to achieve the target exit angle. Iso-angle thresholds may be set as a surgeon preference by the surgeon. The image 1600 depicts 10 degree increments of iso-angles (e.g., 60 degrees, 70 degrees, 80 degrees, 90 degrees, 100 degrees, 110 degrees). The iso-angles feedback 1602 may be overlaid on an image of the patient's bone, a bone model, or both.

In some embodiments, a processor may receive an arthroscopic image of a portion of a patient's body (e.g., knee bone). The processor may determine one or more knee flexion angles that correspond to exit-point placements that may be achievable when placing a tunnel. The processor may overlay the one or more knee flexion angles as graphical elements (e.g., lines, heat map, graph, grid, etc.) over a portion of the arthroscopic image of the portions of the patient's body. In some embodiments, as depicted, the one or more knee flexion angles may be included in the iso-angle feedback 1602 and overlaid as lines each representing a different angle degree of flexion.

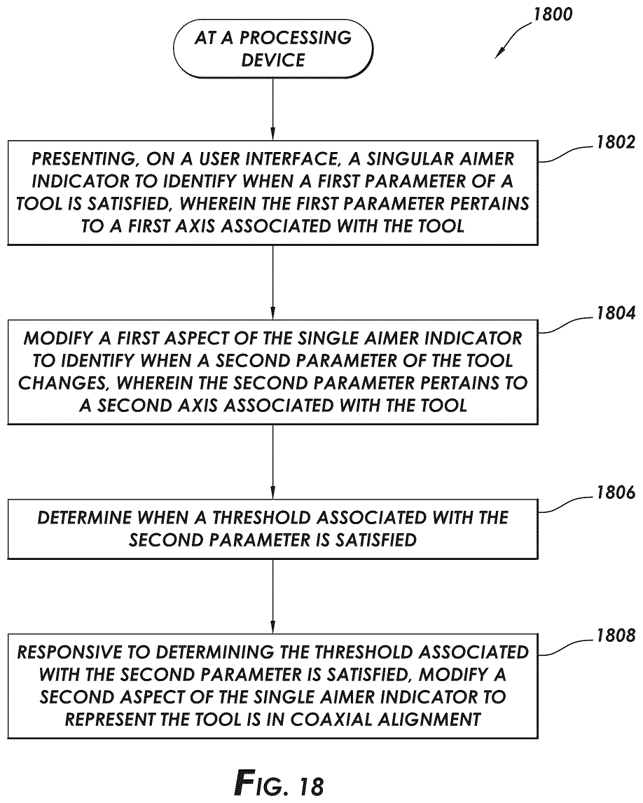

FIG. 17 is another example image 1700 depicting iso-angle feedback 1702, in accordance with at least some embodiments. The depicted embodiment indicates the 90 degree angle as most clearly indicated with a solid line and a numerical display. This angle may be most intuitive for surgeons to know the degree of knee flexion. In some embodiments, any suitable angle may be selected and presented as the most prominent angle by using the solid line and the numerical display or other suitable graphical feature. In the depicted embodiment, other iso-angle flexion lines are indicated by varying the iso-angle line visual indicator, with increments away from the 90 degree line being thinner and more faint for how far away from the mid-line angle the knee is flexed.

In some embodiments, a heat map may be used to display different colors associated with the iso-angles where a certain color is used to identify the target knee flexion angle desired by the surgeon. For example, red may be used to identify the 90 degree knee angle line and different colors may be used to depict the other iso-angle lines as they get farther away from the desired 90 degree knee angle line.

### Software and Hardware

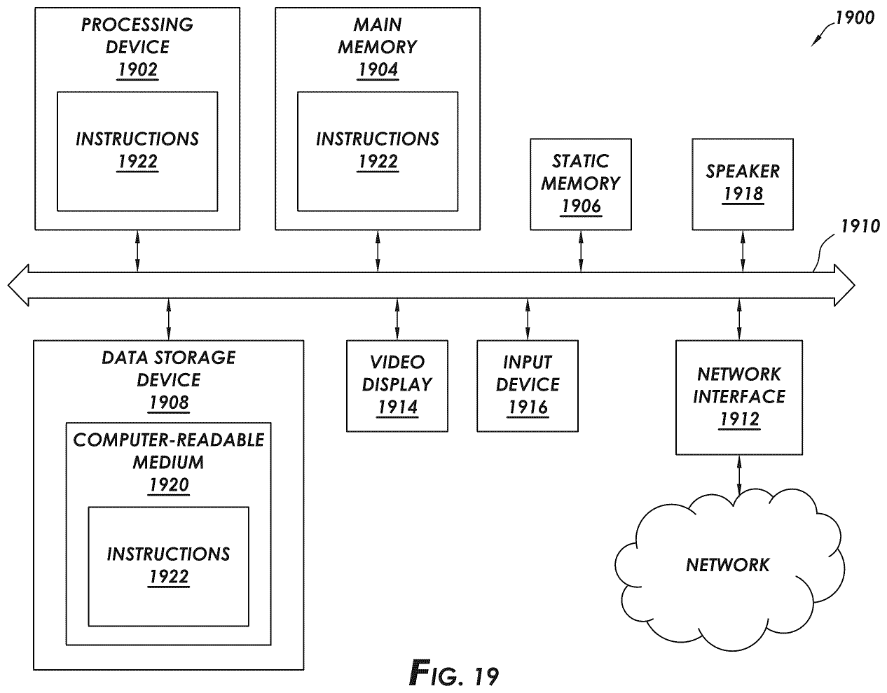

FIG. 18 is an example flow chart of a method for using a single aimer indicator to represent when a tool is in coaxial alignment, in accordance with at least some embodiments. In particular, the methods starts at block 1800. The method may be performed by one or more processors or processing devices, such as included in the surgical controller 418, for example. The method may be implemented in computer instructions stored in one or more memories that are executed by the one or more processors.

At block 1802, the processor may present, on a user interface, a single aimer indicator to identify when a first parameter of a tool is satisfied. The first parameter may pertain to a first axis associated with the tool. The first axis may relate to when a tip of the tool is aligned with a target entry point for a tunnel. In some embodiments, the single aimer indicator may be represented in three-dimensions in real-time or near real-time.

At block 1804, the processor may modify a first aspect of the single aimer indicator to identify when a second parameter of the tool changes. The second parameter pertains to a second axis associated with the tool. The second axis may relate to an angle representing a trajectory of the tool. In some embodiments, the first aspect may include a visual characteristic of the single aimer indicator. For example, the single aimer indicator may include a cross-hair graphical element and the first aspect modified may include increasing or decreasing a thickness of a circle representing the cross-hair graphical element as the alignment of the tool gets closer or farther from being coaxially aligned.

At block 1806, the processor may determine when a threshold associated with the second parameter is satisfied. The threshold may be configurable by a surgeon. The threshold may relate to when the tool tip and/or trajectory of the tool is coaxially aligned within a certain percentage.

At block 1808, responsive to determining the threshold associated with the second parameter is satisfied, the processor may modify a second aspect of the single aimer indicator to represent the tool is in coaxial alignment. The second aspect may include changing a color of the single aimer indicator, flashing a color of the single angle indicator, modifying a border of the single aimer indicator, or some combination thereof.

In some embodiments, a different aimer indicator may be implemented as two scaled bars as described herein. The scaled bars may relate to a parameter for the tool tip distance to a target entry point and to a parameter for an angular difference between a target trajectory and an actual trajectory. The processor may modify an aspect of each of the scaled bars as axial alignment increases or decreases for each of the parameters. For example, the scaled bar representing the tool tip distance to the target entry point may be reduced as the tool tip aligns more closely to the target entry point. When a certain threshold is satisfied, another aspect of the scaled bar may be modified, such as by highlighting the scaled bar, changing a color of the scaled bar, or the like. Also, an aspect of the scaled bar representing the angular difference between the target trajectory and an actual trajectory may be modified as the axial alignment changes. For example, the scaled bar may be reduced as the angular difference is reduced or may be increased (fill up) as the angular difference is increased.

In some embodiments, the processor may determine one or more knee flexion angles that correspond to exit-point output. The processor may provide, in the user interface, an overlay of the one or more determined knee flexion angles as one or more lines on a portion of a bone image (e.g., arthroscopic image), bone model, or both. In some embodiments, each of the lines may include a numerical identifier that specifies the specific degree of the knee flexion angle. In some embodiments, each line may be graphically represented in different manners based on a desired angle or location of a portion of the patient's body. For example, a desired 90 degree knee flexion angle may be represented as a thick line, whereas lines that are farther away from the desired 90 degree knee flexion angle may be dashed lines or thinner lines.

FIG. 19 shows an example computer system 1900. In one example, computer system 1900 may correspond to the surgical controller 418, a tablet device within the surgical room, or any other system that implements any or all the various methods discussed in this specification. The computer system 1900 may be connected (e.g., networked) to other computer systems in a local-area network (LAN), an intranet, and/or an extranet (e.g., device cart 402 network), or at certain times the Internet (e.g., when not in use in a surgical procedure). The computer system 1900 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1900 includes a processing device 1902, a main memory 1904 (e.g., read-only memory (ROM), flash memory, dynamic random access memory (DRAM) such as synchronous DRAM (SDRAM)), a static memory 1906 (e.g., flash memory, static random access memory (SRAM)), and a data storage device 1908, which communicate with each other via a bus 1910.

Processing device 1902 represents one or more general-purpose processing devices such as a microprocessor, central processing unit, or the like. More particularly, the processing device 1902 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1902 may also be one or more special-purpose processing devices such as an application specific integrated circuit (ASIC), a field programmable gate array (FPGA), a digital signal processor (DSP), network processor, or the like. The processing device 1902 is configured to execute instructions for performing any of the operations and steps discussed herein. Once programmed with specific instructions, the processing device 1902, and thus the entire computer system 1900, becomes a special-purpose device, such as the surgical controller 418.

The computer system 1900 may further include a network interface device 1912 for communicating with any suitable network (e.g., the device cart 402 network). The computer system 1900 also may include a video display 1914 (e.g., display device 414), one or more input devices 1916 (e.g., a microphone, a keyboard, and/or a mouse), and one or more speakers 1918. In one illustrative example, the video display 1914 and the input device(s) 1916 may be combined into a single component or device (e.g., an LCD touch screen).

The data storage device 1908 may include a computer-readable storage medium 1920 on which the instructions 1922 (e.g., implementing any methods and any functions performed by any device and/or component depicted described herein) embodying any one or more of the methodologies or functions described herein is stored. The instructions 1922 may also reside, completely or at least partially, within the main memory 1904 and/or within the processing device 1902 during execution thereof by the computer system 1900. As such, the main memory 1904 and the processing device 1902 also constitute computer-readable media. In certain cases, the instructions 1922 may further be transmitted or received over a network via the network interface device 1912.

While the computer-readable storage medium 1920 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

### Clauses

1. A method comprising:
presenting, on a user interface, a single aimer indicator to identify when a first parameter of a tool is satisfied, wherein the first parameter pertains to a first axis associated with the tool;
modifying a first aspect of the single aimer indicator to identify when a second parameter of the tool changes, wherein the second parameter pertains to a second axis associated with the tool;
determining when a threshold associated with the second parameter is satisfied;
responsive to determining the threshold associated with the second parameter is satisfied, modifying a second aspect of the single aimer indicator to represent the tool is in coaxial alignment.

2. The method of any clause herein, wherein the first aspect comprises a visual characteristic of the single aimer indicator.

3. The method of any clause herein, wherein the single aimer indicator comprises a cross-hair graphical element.

4. The method of any clause herein, wherein the single aimer indicator is represented in three-dimensions in real-time or near real-time.

5. The method of any clause herein, further comprising:
providing, in the user interface, an overlay of one or more determined knee flexion angles as one or more lines on a portion of a bone.

6. The method of any clause herein, wherein the first aspect comprises increasing or decreasing a thickness of a circle representing the single aimer indicator.

7. The method of any clause herein, wherein the second aspect comprises changing a color, flashing a color, or both of the single aimer indicator.

8. A surgical controller comprising:
a processor configured to couple to a display device;
a memory coupled to the processor, the memory storing instructions that, when executed by the processor, cause the processor to:
present, on a user interface on the display device, a single aimer indicator to identify when a first parameter of a tool is satisfied, wherein the first parameter pertains to a first axis associated with the tool;
modify a first aspect of the single aimer indicator to identify when a second parameter of the tool changes, wherein the second parameter pertains to a second axis associated with the tool;
determine when a threshold associated with the second parameter is satisfied;
responsive to determining the threshold associated with the second parameter is satisfied, modify a second aspect of the single aimer indicator to represent the tool is in coaxial alignment.

9. The surgical controller of any clause herein, wherein the first aspect comprises a visual characteristic of the single aimer indicator.

10. The surgical controller of any clause herein, wherein the single aimer indicator comprises a cross-hair graphical element.

11. The surgical controller of any clause herein, wherein the single aimer indicator is represented in three-dimensions in real-time or near real-time.

12. The surgical controller of claim 8, wherein the instructions further cause the processor to:
provide, in the user interface, an overlay of one or more determined knee flexion angles as one or more lines on a portion of a bone.

13. The surgical controller of any clause herein, wherein the first aspect comprises increasing or decreasing a thickness of a circle representing the single aimer indicator.

14. The surgical controller of any clause herein, wherein the second aspect comprises changing a color, flashing a color, or both of the single aimer indicator.

15. A tangible, non-transitory computer-readable medium storing instructions that, when executed, cause a processing device to:
present, on a user interface, a single aimer indicator to identify when a first parameter of a tool is satisfied, wherein the first parameter pertains to a first axis associated with the tool;
modify a first aspect of the single aimer indicator to identify when a second parameter of the tool changes, wherein the second parameter pertains to a second axis associated with the tool;
determine when a threshold associated with the second parameter is satisfied;
responsive to determining the threshold associated with the second parameter is satisfied, modify a second aspect of the single aimer indicator to represent the tool is in coaxial alignment.

16. The computer-readable medium of any clause herein, wherein the first aspect comprises a visual characteristic of the single aimer indicator.

17. The computer-readable medium of any clause herein, wherein the single aimer indicator comprises a cross-hair graphical element.

18. The computer-readable medium of any clause herein, wherein the single aimer indicator is represented in three-dimensions in real-time or near real-time.

19. The computer-readable medium of any clause herein, wherein the instructions further cause the processor to:
provide, in the user interface, an overlay of one or more determined knee flexion angles as one or more lines on a portion of a bone.

20. The computer-readable medium of any clause herein, wherein the first aspect comprises increasing or decreasing a thickness of a circle representing the single aimer indicator.

What is claimed is:

1. A method comprising:
presenting, on a user interface, a single aimer indicator to identify when a first parameter of a tool is satisfied, wherein the first parameter pertains to a first axis associated with the tool;
modifying a first aspect of the single aimer indicator to identify when a second parameter of the tool changes, wherein the second parameter pertains to a second axis associated with the tool;
determining when a threshold associated with the second parameter is satisfied;
responsive to determining the threshold associated with the second parameter is satisfied, modifying a second aspect of the single aimer indicator to represent the tool is in coaxial alignment.

2. The method of claim 1, wherein the first aspect comprises a visual characteristic of the single aimer indicator.

3. The method of claim 1, wherein the single aimer indicator comprises a cross-hair graphical element.

4. The method of claim 1, wherein the single aimer indicator is represented in three-dimensions in real-time or near real-time.

5. The method of claim 1, further comprising:
providing, in the user interface, an overlay of one or more determined knee flexion angles as one or more lines on a portion of a bone.

6. The method of claim 1, wherein the first aspect comprises increasing or decreasing a thickness of a circle representing the single aimer indicator.

7. The method of claim 1, wherein the second aspect comprises changing a color, flashing a color, or both of the single aimer indicator.

8. A surgical controller comprising:
a processor configured to couple to a display device;
a memory coupled to the processor, the memory storing instructions that, when executed by the processor, cause the processor to:
present, on a user interface on the display device, a single aimer indicator to identify when a first parameter of a tool is satisfied, wherein the first parameter pertains to a first axis associated with the tool;
modify a first aspect of the single aimer indicator to identify when a second parameter of the tool changes, wherein the second parameter pertains to a second axis associated with the tool;
determine when a threshold associated with the second parameter is satisfied;
responsive to determining the threshold associated with the second parameter is satisfied, modify a second aspect of the single aimer indicator to represent the tool is in coaxial alignment.

9. The surgical controller of claim 8, wherein the first aspect comprises a visual characteristic of the single aimer indicator.

10. The surgical controller of claim 8, wherein the single aimer indicator comprises a cross-hair graphical element.

11. The surgical controller of claim 8, wherein the single aimer indicator is represented in three-dimensions in real-time or near real-time.

12. The surgical controller of claim 8, wherein the instructions further cause the processor to:
provide, in the user interface, an overlay of one or more determined knee flexion angles as one or more lines on a portion of a bone.

13. The surgical controller of claim 8, wherein the first aspect comprises increasing or decreasing a thickness of a circle representing the single aimer indicator.

14. The surgical controller of claim 8, wherein the second aspect comprises changing a color, flashing a color, or both of the single aimer indicator.

15. A tangible, non-transitory computer-readable medium storing instructions that, when executed, cause a processing device to:
present, on a user interface, a single aimer indicator to identify when a first parameter of a tool is satisfied, wherein the first parameter pertains to a first axis associated with the tool;
modify a first aspect of the single aimer indicator to identify when a second parameter of the tool changes, wherein the second parameter pertains to a second axis associated with the tool;
determine when a threshold associated with the second parameter is satisfied;
responsive to determining the threshold associated with the second parameter is satisfied, modify a second aspect of the single aimer indicator to represent the tool is in coaxial alignment.

16. The computer-readable medium of claim 15, wherein the first aspect comprises a visual characteristic of the single aimer indicator.

17. The computer-readable medium of claim 15, wherein the single aimer indicator comprises a cross-hair graphical element.

18. The computer-readable medium of claim 15, wherein the single aimer indicator is represented in three-dimensions in real-time or near real-time.

19. The computer-readable medium of claim 15, wherein the instructions further cause the processor to:
provide, in the user interface, an overlay of one or more determined knee flexion angles as one or more lines on a portion of a bone.

20. The computer-readable medium of claim 15, wherein the first aspect comprises increasing or decreasing a thickness of a circle representing the single aimer indicator.
